(19) United States
(12) Patent Application Publication
(10) Pub. No.: US 2021/0207189 A1
(43) Pub. Date: Jul. 8, 2021

(54) COMPOSITIONS, DEVICES, KITS AND METHODS USEFUL FOR REMOVAL OF PHOSPHOLIPIDS

(71) Applicant: WATERS TECHNOLOGIES CORPORATION, Milford, MA (US)

(72) Inventors: Edouard S. P. Bouvier, Stow, MA (US); Erin E. Chambers, Sutton, MA (US)

(21) Appl. No.: 17/144,422

(22) Filed: Jan. 8, 2021

Related U.S. Application Data
(60) Provisional application No. 62/958,387, filed on Jan. 8, 2020.

(51) Int. Cl. C12Q 1/44; G01N 1/40; G01N 30/14

(52) U.S. Cl. CPC C12Q 1/44; G01N 30/14; G01N 1/4044

(57) ABSTRACT

Novel compositions, devices, kits and methods useful for sample treatment are disclosed herein.

### CROSS-REFERENCE TO RELATED APPLICATION

This application claims priority from and the benefit of U.S. Provisional Patent Application No. 62/958,387 filed on Jan. 8, 2020, the entire content of which is incorporated herein by reference.

### FIELD

The present disclosure relates to compositions, devices, kits and methods that may be used, for example, in conjunction with the processing and/or analysis of a biological sample of interest. In various embodiments, the present disclosure relates to compositions, devices, kits and methods that may be used for removal of matrix molecules, including phospholipids, from a biological sample of interest.

### BACKGROUND

Many biological samples, including biological fluids such as plasma, serum, whole blood, oral fluids, and urine, animal tissue, plant tissue, and certain foods, among others, contain matrix components (i.e., components of a sample other than a target analyte of interest), many of which can interfere with sample analyses, including liquid chromatography-mass spectrometry analysis. For example, when an interference co-elutes with a target analyte, the ionization efficiency of the target analyte during MS analysis can be impacted, either positively or negatively. A decrease in ionization efficiency is called “ion suppression”, while an increase in ionization efficiency is called “ion enhancement”. Because the types and concentrations of matrix interferences can vary significantly from sample to sample, this can result in reduced precision and accuracy when quantifying target analytes.

Endogenous phospholipids present in biological samples such as plasma and serum samples, among others, are a broad class of interferences that have significant impact on the ionization efficiency of target analytes. Phospholipids are comprised of a polar “head” group, and a hydrophobic “tail” group. As can be seen from the phospholipid formula shown in FIG. 1 (corresponding to phosphatidylcholine), the hydrophobic tail is made up of two fatty acyl chains and the polar head group includes a phosphate group, a glycerol residue, and a variable moiety (for phosphatidylcholine, the variable moiety is choline).

Phospholipids are a key component of the lipid bilayer that comprises cellular membranes. There are many different chemical entities that encompass the class of phospholipids, with the primary type in human plasma being phosphatidylcholine. Phospholipid composition in plasma has been described in several references including, for example, S. Bradamante, et al, “An Alternative Expeditious Analysis of Phospholipid Composition in Human Blood Plasma by 31P-NMR Spectroscopy”, Anal. Biochem. 185, 299-303 (1990) and J. L. Little et al, “Liquid chromatography-mass spectrometry/mass spectrometry method development for drug metabolism studies: Examining lipid matrix ionization effects in plasma”, J. Chromatogr. B, 833, 219-230 (2006).

When injected onto a chromatography column, for example a reversed-phase chromatography column, phospholipids can elute throughout a large portion of the chromatographic separation. As a result, the quantitation of any analytes that co-elute during this broad window will be impacted. Phospholipids can also lead to column and instrument fouling during LC-MS analysis.

Several products, including various solid-phase extraction (SPE) products, have been commercialized which can selectively capture and remove interfering phospholipids from a sample prior to chromatographic analysis, and thereby providing for better quantitation of target analytes. For example, Waters Corporation has commercialized Oasis™ Prime HLB and Oasis™ MCX Elution plates for this purpose. The HLB plates retain phospholipids by hydrophobic interaction, while the MCX plates capture primarily by ion exchange. Generally, the choice of SPE chemistry to use depends on the type of analytes one is analyzing for. The present disclosure provides a more universal means for phospholipid removal which is less dependent on the specific properties of the target analytes.

### SUMMARY

In various aspects, the present disclosure describes chromatographic methods comprising: (a) contacting a biological sample that comprises at least one phospholipid and at least one target analyte with a phospholipase enzyme in aqueous solution such that the phospholipid is enzymatically digested and (b) subjecting the digested sample to liquid chromatography to form an eluent comprising the at least one target analyte.

In various embodiments, which may be used with the preceding aspects, the sample fluid may comprise a biological sample selected from a whole blood sample, a plasma sample, a serum sample, a food sample, and a food extract sample.

In various embodiments, which may be used with any of the preceding aspects and embodiments, the phospholipase may be selected from Phospholipase A1, Phospholipase A2, Phospholipase B and Phospholipase C.

In various embodiments, which may be used with any of the preceding aspects and embodiments, the liquid chromatography may be selected from reversed-phase chromatography (RPC), hydrophilic-interaction chromatography (HILIC), hydrophobic-interaction chromatography (HIC), ion-exchange chromatography (IEC), and normal-phase chromatography (NPC).

In various embodiments, which may be used with any of the preceding aspects and embodiments, the methods may further comprise performing mass spectrometry analysis on at least a portion of the eluent.

In various embodiments, which may be used with any of the preceding aspects and embodiments, the biological sample may be contacted with the phospholipase enzyme online with the liquid chromatography, or the biological sample may be contacted with the phospholipase enzyme offline prior to the liquid chromatography.

In various embodiments, which may be used with any of the preceding aspects and embodiments, the phospholipase enzyme may be a free enzyme.

In various embodiments, which may be used with any of the preceding aspects and embodiments, the phospholipase enzyme may be attached to a solid support.

In various aspects, the present disclosure is directed to enzymatic supports comprising a solid support and a phospholipase enzyme attached to the solid support.

In various embodiments, the phospholipase enzyme may be selected from Phospholipase A1, Phospholipase A2, Phospholipase B and Phospholipase C.

In various embodiments, which may be used with any of the preceding aspects and embodiments, the solid support may be selected from a particle, a fibrous material, a monolithic structure, an aerogel and a membrane.

In various embodiments, which may be used with any of the preceding aspects and embodiments, the solid support may be a porous solid support.

In various embodiments, which may be used with any of the preceding aspects and embodiments, the solid support material may be selected from an organic material, an inorganic material, and an organic-inorganic hybrid material.

In various aspects, the present disclosure pertains to kits which comprise a enzymatic support in accordance with any of the preceding aspects and embodiments and an enzymatic support housing.

In various embodiments, which may be used with any of the preceding aspects and embodiments, the enzymatic support housing may comprise a chamber for holding the enzymatic support, an inlet and an outlet.

In various embodiments, which may be used with any of the preceding aspects and embodiments, the enzymatic support housing may be selected from a syringe, a cartridge, a column, a multi-well device, and a pipette tip.

In various embodiments, which may be used with any of the preceding aspects and embodiments, the enzymatic support may be provided separate from the enzymatic support housing or the enzymatic support may be disposed in the enzymatic support housing.

In various aspects, the present disclosure pertains to kits for the removal of phospholipids from a biological sample that comprise (a) phospholipase enzyme in accordance with any of the preceding aspects and embodiments and (b) a chromatographic sorbent.

In various embodiments, the phospholipase enzyme may be a free enzyme, or the phospholipase enzyme may be attached to a solid support.

In various embodiments, which may be used with any of the preceding aspects and embodiments, the solid support material may be selected from an organic material, an inorganic material, and an organic-inorganic hybrid material.

In various embodiments, which may be used with any of the preceding aspects and embodiments, the kits may further comprise an enzymatic support housing.

In various embodiments, which may be used with any of the preceding aspects and embodiments, enzymatic support housing may comprise a chamber for holding the enzymatic support, an inlet and an outlet.

In various embodiments, which may be used with any of the preceding aspects and embodiments, the enzymatic support housing may be selected from a syringe, a cartridge, a column, a multi-well device, and a pipette tip.

In various embodiments, which may be used with any of the preceding aspects and embodiments, the enzymatic support may be provided separate from the enzymatic support housing, or the enzymatic support may be disposed in the enzymatic support housing.

In various embodiments, which may be used with any of the preceding aspects and embodiments, the chromatographic sorbent may be selected from a reversed-phase chromatographic sorbent, a hydrophilic-interaction chromatographic sorbent, a hydrophobic-interaction chromatographic sorbent, an ion-exchange chromatographic sorbent and a normal-phase chromatographic sorbent.

In various embodiments, which may be used with any of the preceding aspects and embodiments, the chromatographic sorbent may be provided in a chromatographic sorbent housing.

In various embodiments, which may be used with any of the preceding aspects and embodiments, the chromatographic sorbent housing may comprise a chamber for holding the chromatographic support, an inlet and an outlet.

In various embodiments, which may be used with any of the preceding aspects and embodiments, the chromatographic sorbent housing may be selected from a syringe, a cartridge, a column, a multi-well device, and a pipette tip.

In various embodiments, which may be used with any of the preceding aspects and embodiments, the chromatographic sorbent may be provided separate from the chromatographic sorbent housing, or the chromatographic sorbent may be disposed in the chromatographic sorbent housing.

These and other aspects, as well as numerous embodiments and advantages associated with the methods, compositions, devices and kits described in the present disclosure will become immediately apparent to those of ordinary skill in the art upon review of the detailed description and claims to follow.

### BRIEF DESCRIPTION OF THE DRAWINGS

FIG. 1 is a schematic illustration of a phospholipid, in accordance with the prior art.

FIG. 2 is a schematic illustration of various phospholipase cleavage sites within a phospholipid, in accordance with the prior art.

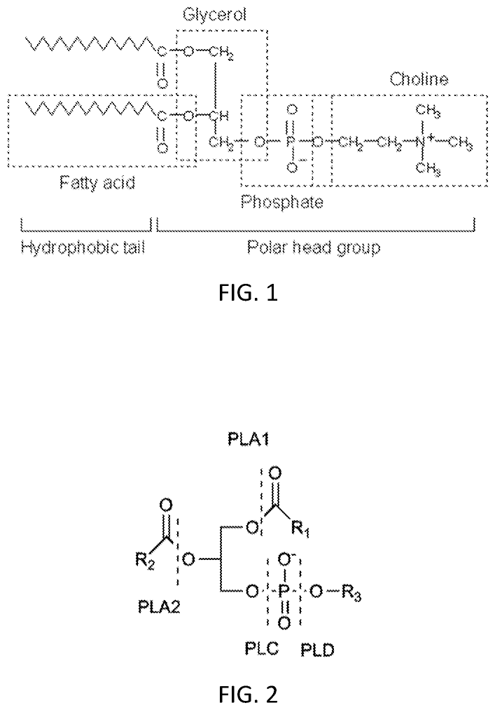

### DETAILED DESCRIPTION

One of the challenges associated with removing phospholipid interferences from biological samples is the fact that phospholipids have an ionizable polar head and a hydrophobic tail, which can cause them to be retained on a chromatographic column by multiple retention mechanisms. This can cause substantial peak tailing, making co-elution of a target analyte of interest with phospholipid species more likely, in which case ion suppression can occur, among other possible deleterious effects.

In the present disclosure, phospholipids are enzymatically cleaved into multiple distinct species, including species associated with the polar “head” portion and species associated with the hydrophobic “tail” portion. In this manner, the mechanism for chromatographic retention is simplified, which in turn simplifies phospholipid removal. For example, in the case of a reversed phase column, species associated with the polar “head” portion becomes poorly retained on the column, whereas species associated with the hydrophobic “tail” portion are strongly retained. Once the phospholipids are cleaved into multiple species, peak tailing may be significantly reduced or eliminated, and these species eluted in separate narrow chromatographic windows. As a result, target analytes may thus be more readily separated from phospholipid interferences, resulting in substantially improved quantitation precision and accuracy.

In various embodiments, the present disclosure describes methods of treating biological samples that contain at least one phospholipid and at least one potential target analyte, which methods include contacting the biological sample with a phospholipase enzyme in aqueous solution such that the at least one phospholipid is enzymatically digested. Exemplary biological samples for use in the present disclosure include any biological sample that contains or potentially contains phospholipids, such as biological fluids (e.g., whole blood samples, blood plasma samples, serum samples, oral fluids, urine, etc.), biological tissues, biological matrices, cells (e.g., one or more types of cells), cell culture supernatants, foods that contain phospholipids (e.g., meats, whole grains, legumes, eggs, etc.), and food extracts.

After the biological sample is contacted with the phospholipase enzyme and phospholipids in the biological sample are enzymatically digested, the digested sample may be subjected to further processing and/or analysis.

In various embodiments, the digested sample is subjected to chromatographic separation to create an eluent fraction containing (or potentially containing) at least one target analyte, which contains low levels of phospholipids or digested portions thereof. Chromatographic separation processes for use in the present disclosure include liquid chromatography (LC) methods, including high performance liquid chromatography (HPLC) and ultra-high performance liquid chromatography (UHPLC). Various types of liquid chromatography are known which may be used in the present disclosure including reversed-phase chromatography (RPC), hydrophilic-interaction chromatography (HILIC), hydrophobic-interaction chromatography (HIC), ion-exchange chromatography (IEC), and normal-phase chromatography (NPC), among others. In certain cases, the digested sample may be evaporated to dryness, and then reconstituted in another solution before being injected into a liquid chromatography system.

In various embodiments, the methods of the present disclosure further comprise additional processing of at least a portion of eluent from the liquid chromatography separation process (e.g., an eluent fraction containing, or potentially containing, at least one target analyte of interest), for example, to identify, quantify, or otherwise process the one or more target analytes.

In certain beneficial embodiments, mass spectrometry analysis is performed on at least a portion of eluent from the liquid chromatography separation process. Particular examples of mass spectrometry (MS) include electrospray ionization mass spectrometry (ESI-MS), matrix-assisted laser desorption/ionization mass spectrometry (MALDI-MS), and time-of-flight mass spectrometry (TOFMS), among others. Alternatively or in addition, at least a portion of the eluent from the liquid chromatography separation process may be subjected to other analytical techniques including nuclear magnetic resonance, infrared analysis or ultraviolet analysis, among others.

Specific examples of phospholipase enzymes that can be employed to enzymatically digest phospholipids in a biological sample of interest include (a) phospholipase A1 (PLA1), which cleaves the SN-1 acyl chain (where SN refers to stereospecific numbering) of the phospholipid (e.g., releasing a 2-acyl-lysophospholipid and a fatty acid), (b) phospholipase A2 (PLA2), which cleaves the SN-2 acyl chain of the phospholipid (e.g., releasing a 1-acyl-lysophospholipid and a fatty acid), (c) phospholipase B (PLB), which cleaves both SN-1 and SN-2 acyl chains of the phospholipid (e.g., releasing two fatty acids and a glycerophospho-compound, for instance, a glycerophosphosphoryl base) and thus has a combination of PLA1 and PLA2 activities, (d) phospholipase C (PLC), which cleaves the phospholipid, for example, releasing a diacylglycerol and a phosphate-containing head group, and (e) phospholipase D (PLD), which cleaves the phospholipid, for example, releasing phosphatidic acid and an alcohol.

Cleavage positions for each of these phospholipases is shown with dashed lines in FIG. 2, where R₁ is a saturated or unsaturated alkyl group, for example, having from 4 to 28 carbon atoms, where R₂ is a saturated or unsaturated alkyl group, for example, having from 4 to 28 carbon atoms, and where R₃ is hydrogen or an organic moiety, for example, ethanolamine, choline, serine, glycerol, phosphatidylglycerol or inositol, among others.

In various embodiments, the phospholipase enzyme that is used to digest the biological sample may be a free enzyme that is contacted with the biological sample.

In various embodiments, the phospholipase enzyme that is used to digest the biological sample may be attached to a solid support (the solid support with attached phospholipase enzyme is referred to herein as an “enzymatic support”). In many of these embodiments, the phospholipase enzyme is covalently attached to the solid support.

Solid supports may be provided in any suitable shape and size, for example, being provided in the form of a particle (e.g., having a diameter ranging from about 2 μm to about 100 μm in diameter, a fibrous material, a monolithic structure (e.g., a slab, disc, container wall, etc.), an aerogel, a membrane, and so forth.

Suitable solid supports for use in conjunction with the present disclosure may be non-porous or porous. Suitable porous solid support materials include fully porous and superficially porous solid support materials. Suitable porous solid support materials may have pore sizes that are sufficiently large to hold an attached phospholipase enzyme and to allow the phospholipids to diffuse in and out of the pores. For example, pore sizes may range from about 10 nm to about 200 nm, among other values.

Materials for use in the present disclosure as solid support materials include any suitable material to which a phospholipase enzyme may be attached. Examples of solid support materials include organic materials, inorganic materials, and organic-inorganic hybrid materials.

Particular examples of inorganic materials include, for example, glass, silica-based materials (including silica), metal-oxide-based materials (e.g., alumina-based materials, titania-based materials, zirconia-based materials, etc.), carbon-based materials (e.g., carbon-based inorganic materials including pure carbon, carbon-based organic materials, and carbon-based organic-inorganic hybrid materials) and metal-based particles (e.g., gold or gold-coated particles, which may be used for attachment of additional species via sulfhydryl linkages).

In certain beneficial embodiments, silica-based materials, including inorganic materials (silica) and inorganic-organic hybrid materials, may be formed by hydrolytically condensing one or more organosilane compounds, which may, for example, comprise one or more alkoxysilane compounds, for instance, one or more tetraalkoxysilane compounds, one or more alkylalkoxysilane compounds, or a combination of one or more tetraalkoxysilane compounds and one or more alkylalkoxysilane compounds. Specific examples of alkoxysilane compounds include, for instance, tetraalkoxysilane compounds (e.g., tetramethoxysilane (TMOS), tetraethoxysilane (TEOS), etc.), alkylalkoxysilane compounds such as alkyltrialkoxysilanes (e.g., methyl trimethoxysilane, methyl triethoxysilane (MTOS), ethyl triethoxysilane, etc.) and bis(trialkoxysilyl)alkanes (e.g., bis(trimethoxysilyl)methane, bis(trimethoxysilyl)ethane, bis(triethoxysilyl)methane, bis(triethoxysilyl)ethane (BTE), etc.), as well as combinations of any two or more of the foregoing tetraalkoxysilane compounds and alkylalkoxysilane compounds.

Thus, in certain embodiments, the silica-based materials may be prepared from two monomers: (a) a tetraalkoxysilane such as TMOS or TEOS and (b) an alkylalkoxysilane such as MTOS or a bis(trialkoxysilyl)alkane such as BTEE. When BTEE is employed as a monomer, the resulting materials are organic-inorganic hybrid materials, which are sometimes referred to as ethylene bridged hybrid (BEH) materials and can offer various advantages over conventional silica-based materials, including chemical and mechanical stability.

In certain beneficial embodiments, the solid support comprises an organic material in the form of a polymer or an organic-inorganic hybrid material that comprises a polymer. In particular embodiments, the polymer may be, for example, a polysaccharide such as agarose, a organic polymer such as a methacrylate polymer or copolymer, a styrene polymer or copolymer, divinylbenzene polymer or copolymer (e.g., a styrene-divinylbenzene copolymer), or an organic copolymer comprising a hydrophilic monomer and a hydrophobic monomer.

With regard to the organic copolymer that comprises at least one hydrophobic organic monomer and at least one hydrophilic organic monomer, in certain embodiments, the hydrophilic organic monomer may be selected from organic monomers having an amide group, organic monomers having an ester group, organic monomers having a carbonate group, organic monomers having a carbamate group, organic monomers having a urea group, organic monomers having a hydroxyl group, and organic monomers having nitrogen-containing heterocyclic group, among other possibilities. Specific examples of hydrophilic organic monomers include, for example, 2-vinylpyridine, 3-vinylpyridine, 4-vinylpyridine, N-vinylpyrrolidone, N-vinyl-piperidone, N-vinyl caprolactam, lower alkyl acrylates (e.g., methyl acrylate, ethyl acrylate, etc.), lower alkyl methacrylates (e.g., methyl methacrylate, ethyl methacrylate, etc.), vinyl acetate, acrylamide or methacrylamide, hydroxypolyethoxy allyl ether, ethoxy ethyl methacrylate, ethylene glycol dimethacrylate, or diallyl maleate, as well as combinations of the foregoing. In certain beneficial embodiments, the hydrophilic organic monomer may be a monomer having the following formula,

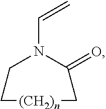

where n ranges from 1-3 (i.e., N-vinyl pyrrolidone, N-vinyl-2-piperidinone or N-vinyl caprolactam).

In certain embodiments, the hydrophobic monomer may comprise a C₆-C₁₈ monocyclic or multicyclic carbocyclic group, e.g., a phenyl group or a phenylene group. Specific examples of hydrophobic monomers include, for example, monofunctional and multifunctional aromatic monomers such as styrene and divinylbenzene, monofunctional and multifunctional olefin monomers such as ethylene, propylene or butylene, polycarbonate monomers, ethylene terephthalate, monofunctional and multifunctional fluorinated monomers such as fluoroethylene, 1,1-difluoroethylene), tetrafluoroethylene, chlorotrifluoroethylene, hexafluoropropylene, perfluoropropylvinylether, or perfluoromethylvinylether, monofunctional or multifunctional acrylate monomers having a higher alkyl group, monofunctional or multifunctional acrylate monomers having a C₆-C₁₈ saturated, unsaturated or aromatic carbocyclic group, monofunctional or multifunctional methacrylate monomers having a higher alkyl group, monofunctional or multifunctional methacrylate monomers having a C₆-C₁₈ saturated, unsaturated or aromatic carbocyclic group, as well as combinations of the foregoing.

In certain embodiments, the organic copolymer may comprise n-vinyl pyrrolidone or n-vinyl caprolactam as a hydrophilic organic monomer and divinylbenzene as a hydrophobic organic monomer. Specific examples of such copolymers include Oasis™ type polymers particles available from Water Corporation, including Oasis HLB™ particles, among others.

In some embodiments, the enzymatic support may be provided in conjunction with a suitable housing (referred to herein as an “enzymatic support housing”).

The enzymatic support and the enzymatic support housing may be supplied independently (e.g., each provided in individual packaging), or the enzymatic support may be pre-packaged in the enzymatic support housing, for example, in the form of a packed bed, among other possibilities.

Enzymatic support housings for use in accordance with the present disclosure typically include a chamber for accepting and holding the enzymatic support. In various embodiments, the enzymatic support housing may be provided with an inlet and an outlet.

Materials that may be used for construction of the enzymatic support housing include inorganic materials, for instance, metals such as stainless steel and ceramics such as glass, as well as synthetic polymeric materials such as polyethylene, polypropylene, polyether ether ketone (PEEK), and polytetrafluoroethylene, among others. Housing materials may be coated or functionalized to decrease adsorption interactions of target phospholipids. Coating/functionalization may be performed, for example, by chemical vapor deposition, atomic layer deposition, or wet chemistry. In certain embodiments where monoliths are used, the housing materials may be surface functionalized with appropriate attachment groups to improve adhesion of monolith to the housing. For example, a vinyl functionalization could be employed to improve adhesion of methacrylate or divinylbenzene based monoliths.

In certain embodiments, the enzymatic support housing may include one or more filters which act to hold the enzymatic support in an enzymatic support housing. Exemplary filters may be, for example, in a form of membrane, screen, frit or spherical porous filter.

In certain embodiments, a solution received in the enzymatic support housing may flow into or onto the enzymatic support spontaneously, for example, capillary action. In certain embodiments, flow through the enzymatic support in the enzymatic support housing may be generated by external forces, such as gravity or centrifugation, or by applying a vacuum to an outlet of the enzymatic support housing or positive pressure to an inlet of the enzymatic support housing.

Some specific examples of enzymatic support housings for use in the present disclosure include, for example, syringes, injection cartridges, columns, multi-well enzymatic support housings such as a 4 to 8-well rack, a 4 to 8-well strip, or a 48 to 96-well plate, a spin tube or a spin container.

As previously indicated, after the biological sample is contacted with the phospholipase enzyme such that phospholipids in the biological sample are enzymatically digested, the digested sample may be subjected to further processing, for example, to isolate, identify, quantify, or otherwise process one or more target analytes.

In various embodiments, the digested sample may be subjected to chromatographic separation.

In certain embodiments, the digestion step may be performed offline prior chromatographic separation. In certain embodiments, the digestion step may be performed online in conjunction with chromatographic separation (e.g., by employing a column packed with phospholipase enzyme immobilized on a solid support).

Chromatographic separation methods for use in the present disclosure include liquid chromatography methods such as reversed-phase chromatography (RPC), hydrophilic-interaction chromatography (HILIC), hydrophobic-interaction chromatography (HIC), ion-exchange chromatography (IEC), and normal-phase chromatography (NPC), among others.

Various sorbent materials can be used in conjunction with the preceding chromatographic separation methods.

In some embodiments, the chromatographic sorbent materials may be selected, for example, organic materials, inorganic materials, and organic-inorganic hybrid materials. Particular examples of sorbent materials include, for example, silica-based materials such as those described above, metal-oxide-based materials (e.g., alumina-based materials, titania-based materials, zirconia-based materials, etc.), and carbon-based materials. Particular examples of sorbent materials further include organic materials in the form of an organic polymer or an organic-inorganic hybrid material that comprises an organic polymer. Examples of organic polymers are set forth above, and include organic copolymers that comprise a hydrophilic monomer and a hydrophobic monomer described above (e.g., Oasis HLB™ sorbent particles).

In various embodiments, the preceding chromatographic sorbent materials may be provided with variety of covalently bonded groups to modify the chromatographic character of the sorbent. Examples of such groups include bonded alkyl groups including straight chain C₂-C₁₈-alkyl groups or branched chain C₃-C₁₈-alkyl groups, such as ethyl (C₂), butyl (C₄), octyl (C₈), or octadecyl (C₁₈) groups, bonded C₆-C₁₈ monocyclic or multicyclic, saturated, unsaturated or aromatic carbocyclic groups, such as phenyl groups or a phenylene groups, bonded primary and/or secondary amine groups including aminoalkyl groups such as aminopropyl groups, bonded cyano groups including cyanoalkyl groups such as cyanopropyl groups, bonded amide groups, bonded carbamate groups, bonded diol groups, bonded polyol groups, bonded zwitterionic groups, and bonded ion-exchange groups, including strong cation exchange groups (e.g., strong anionic groups such as sulfonate groups), strong anion exchange groups (e.g., strong cationic groups such as quaternary ammonium groups), weak cation exchange groups (e.g., weak anionic groups such as carboxyl groups), or weak anion exchange groups (e.g., weak cationic groups such as primary, secondary or tertiary amine groups).

In embodiments the chromatographic sorbent material comprises an organic polymer or copolymer, the organic polymer or copolymer may further comprise an organic monomer that comprises one or more anionic groups and/or an organic monomer that comprises one or more cationic groups, for example, to provide the sorbent with ion exchange characteristics. The organic monomer that comprises one or more anionic groups and/or the organic monomer that comprises one or more cationic groups may be incorporated into the organic polymer or copolymer by copolymerization at the time the organic polymer or copolymer is formed, or may be incorporated into the organic polymer or copolymer by derivatization of the organic polymer or copolymer subsequent to formation. In either case, the result may be, for example, an organic polymer or copolymer comprising an organic monomer that provides strong cation exchange characteristics, an organic polymer or copolymer comprising an organic monomer that provides weak cation exchange characteristics, an organic polymer or copolymer comprising an organic monomer that provides strong anion exchange characteristics, and/or an organic polymer or copolymer comprising an organic monomer that provides weak anion exchange characteristics. For example, the organic polymer or copolymer may comprise an organic monomer that provides strong cation exchange characteristics, in particular, an organic monomer having one or more anionic groups that maintain a negative charge over a wide pH range such as, for instance, sulfonate groups. In particular embodiments, the organic monomer may be a sulfonyl-substituted divinyl benzene monomer. As another example, the organic polymer or copolymer may comprise an organic monomer that provides strong anion exchange characteristics, in particular, an organic monomer having one or cationic groups that maintain a positive charge over a wide pH range such as quaternary ammonium groups, for instance, an organic monomer that comprises one or more —R₁—N⁺R₂R₃R₄ groups, where R₁ is an alkylene group, typically, a C₁-C₈ alkylene group, and R₂, R₃ and R₄ may be the same or different and are alkyl groups, typically, C₁-C₈ alkyl groups. In particular embodiments, the organic monomer may be a quaternary-ammonium-substituted divinyl benzene monomer. As another example, the organic polymer or copolymer may comprise an organic monomer that provides weak cation ion exchange characteristics, in particular, an organic monomer having one or more anionic groups that become neutralized at lower pH levels such as, for instance, carboxylate groups. In particular embodiments, the organic monomer may be a carboxyl-substituted divinyl benzene monomer. As another example, the organic polymer or copolymer may comprise an organic monomer that provides weak anion exchange characteristics, in particular, an organic monomer having one or cationic groups that become neutralized at higher pH levels such as, for instance, primary, secondary or tertiary amine groups, for example, piperazinyl, N-methylpiperazinyl, pyrazinyl, piperidinyl, morpholino, pyrrolidinyl, quinolinyl, pyridyl, pyrimidyl, pyrrolyl, or indolyl groups or phosphate (3-) or carbonate (2-) groups. In particular embodiments, the organic monomer may be a piperazinyl-substituted divinyl benzene monomer. Particular examples of organic-polymer-based sorbents having ion exchange characteristics include Oasis MCX™ strong cation exchange sorbent particles, Oasis WCX™ weak cation exchange sorbent particles, Oasis MAX™ strong anion exchange sorbent particles, and Oasis WAX™ weak anion exchange sorbent particles, among others.

In certain embodiments, where the chromatographic sorbent is an organic-polymer-based sorbent, the organic polymer or copolymer may further comprise an organic monomer that comprises one or more zwitterionic groups. Particular examples of organic monomers that comprise zwitterionic groups can be found, for example, in André Laschewsky, “Structures and Synthesis of Zwitterionic Polymers,” Polymers 2014, 6(5), 1544-1601; doi:10.3390/polym6051544 and include N-(2-methacryloyloxy)ethyl-N,N-dimethylammonio propanesulfonate (SPE), N-(3-methacryloylimino)propyl-N,N-dimethylammonio propanesulfonate (SPP), 2-(methacryloyloxy)ethylphosphatidylcholine (MPC), and 3-(2′-vinyl-pyridinio)propanesulfonate (SPV), which are commercially available.

In various embodiments, sorbents for use in conjunction with the present disclosure may be provided in conjunction with a suitable housing (referred to herein as a “sorbent housing”).

The sorbent and the sorbent housing may be supplied independently, or the sorbent may be pre-packaged in the sorbent housing, for example, in a packed bed.

Sorbent housings for use in accordance with the present disclosure commonly include a chamber for accepting and holding sorbent. In various embodiments, the sorbent housings may be provided with an inlet and an outlet.

Suitable construction materials for the sorbent housings include inorganic materials, for instance, metals such as stainless steel and ceramics such as glass, as well as synthetic polymeric materials such as polyethylene, polypropylene, polyether ether ketone (PEEK), and polytetrafluoroethylene, among others.

In certain embodiments, the sorbent housings may include one or more filters which act to hold the sorbent in a sorbent housing. Exemplary filters may be, for example, in a form of membrane, screen, frit or spherical porous filter.

In certain embodiments, a solution received in the sorbent housing may flow into the sorbent spontaneously, for example, capillary action. In certain embodiments, the flow may be generated through the sorbent by external forces, such as gravity or centrifugation, or by applying a vacuum to an outlet of the sorbent housing or positive pressure to an inlet of the sorbent housing.

Specific examples of sorbent housings for use in the present disclosure include, for example, a syringe, an injection cartridge, a column, a multi-well device such as a 4 to 8-well rack, a 4 to 8-well strip, a 48 to 96-well plate, a 96 to 384-well micro-elution plate, micro-elution tip devices, including a 4 to 8-tip micro-elution strip, a 96 to 384-micro-elution tip array, a single micro-elution pipet tip, a thin layer plate, a microtiter plate, a spin tube or a spin container.

Other aspects of the present disclosure pertain to chromatographic kits that comprise a phospholipase enzyme and a chromatographic sorbent. In some embodiments, the phospholipase enzyme may be in the form of a free enzyme that is supplied in a suitable container. In some embodiments, the phospholipase enzyme may be provided in the form of an enzymatic support that comprises the phospholipase enzyme immobilized on a solid support material, such as those described above. In some embodiments, the enzymatic support is supplied in conjunction with a suitable enzymatic support housing as describe above. For example, the enzymatic support may be pre-packaged in the enzymatic support housing or may be provided separately from the enzymatic support housing (e.g., in a suitable container). Chromatographic sorbents include those described above. In some embodiments, the chromatographic sorbent may be supplied in conjunction with a suitable sorbent housing as described above. For example, the chromatographic sorbent may be pre-packaged in the sorbent housing or may be provided separately from the sorbent housing (e.g., in a suitable container).

In certain embodiments, in addition to the phospholipase enzyme and the chromatographic sorbent, the kits may further comprise one or more of the following: (a) a collection plate or collection vial, (b) a cap mat, (c) calibration and reference standards, (d) instructions for use, and (e) identification tagging for each component, which may include passive tags, such as RFID tags, for tracking the components.

1. A method comprising:
contacting a biological sample that comprises at least one phospholipid and at least one target analyte with a phospholipase enzyme in aqueous solution such that the phospholipid is enzymatically digested; and
subjecting the digested sample to liquid chromatography to form an eluent comprising the at least one target analyte.

2. The method of claim 1, wherein the sample fluid comprises a biological sample selected from a whole blood sample, a plasma sample, a serum sample, a food sample, and a food extract sample.

3. The method of claim 1, wherein the phospholipase enzyme is selected from Phospholipase A1, Phospholipase A2, Phospholipase B and Phospholipase C.

4. The method of claim 1, wherein the liquid chromatography is selected from reversed-phase chromatography (RPC), hydrophilic-interaction chromatography (HILIC), hydrophobic-interaction chromatography (HIC), ion-exchange chromatography (IEC), and normal-phase chromatography (NPC).

5. The method of claim 1, further comprising performing mass spectrometry analysis on at least a portion of the eluent.

6. The method of claim 1, wherein the biological sample is contacted with the phospholipase enzyme online with the liquid chromatography or wherein the biological sample is contacted with the phospholipase enzyme offline prior to the liquid chromatography.

7. The method of claim 1, wherein the phospholipase enzyme is a free enzyme.

8. The method of claim 1, wherein the phospholipase enzyme is attached to a solid support.

9. An enzymatic support comprising a solid support and a phospholipase enzyme attached to the solid support.

10. The enzymatic support of claim 9, wherein the phospholipase enzyme is selected from Phospholipase A1, Phospholipase A2, Phospholipase B and Phospholipase C.

11. The enzymatic support of claim 9, wherein the solid support is selected from a particle, a fibrous material, a monolithic structure, an aerogel and a membrane.

12. The enzymatic support of claim 9, wherein the solid support is a porous solid support.

13. The enzymatic support of claim 9, wherein the solid support material is selected from an organic material, an inorganic material, and an organic-inorganic hybrid material.

14. A kit comprising the enzymatic support of claim 9 and an enzymatic support housing.

15. The kit of claim 14, wherein the enzymatic support housing comprises a chamber for holding the enzymatic support, an inlet and an outlet.

16. The kit of claim 14, wherein the enzymatic support housing is selected from a syringe, a cartridge, a column, a multi-well device, and a pipette tip.

17. The kit of claim 14, wherein the enzymatic support is provided separate from the enzymatic support housing or is disposed in the enzymatic support housing.

18. A kit for removal of phospholipids from a biological sample comprising (a) phospholipase enzyme and (b) a chromatographic sorbent.

19. The kit of claim 18, wherein the phospholipase enzyme is a free enzyme.

20. The kit of claim 18, comprising an enzymatic support comprising a solid support and the phospholipase enzyme attached to the solid support.

21. The kit of claim 20, wherein the solid support material is selected from an organic material, an inorganic material, and an organic-inorganic hybrid material.

22. The kit of claim 20, further comprising an enzymatic support housing.

23. The kit of claim 22, wherein the enzymatic support housing comprises a chamber for holding the enzymatic support, an inlet and an outlet.

24. The kit of claim 22, wherein the enzymatic support housing is selected from a syringe, a cartridge, a column, a multi-well device, and a pipette tip.

25. The kit of claim 22, wherein the enzymatic support is provided separate from the enzymatic support housing or disposed in the enzymatic support housing.

26. The kit of claim 18, wherein the chromatographic sorbent is selected from a reversed-phase chromatographic sorbent, a hydrophilic-interaction chromatographic sorbent, a hydrophobic-interaction chromatographic sorbent, an ion-exchange chromatographic sorbent and a normal-phase chromatographic sorbent.

27. The kit of claim 26, further comprising a chromatographic sorbent housing.

28. The kit of claim 27, wherein the chromatographic sorbent housing comprises a chamber for holding the chromatographic support, an inlet and an outlet.

29. The kit of claim 27, wherein the chromatographic sorbent housing is selected from a syringe, a cartridge, a column, a multi-well device, and a pipette tip.

30. The kit of claim 27, wherein the chromatographic sorbent is provided separate from the chromatographic sorbent housing or disposed in the chromatographic sorbent housing.
